(19) United States
(12) Patent Application Publication
(10) Pub. No.: US 2012/0143058 A1
(43) Pub. Date: Jun. 7, 2012

(54) PROPAGATION-MEDIUM-MODIFICATION-BASED REVERBERATED-SIGNAL ELIMINATION

(75) Inventors: Jeffry E. Powers, Bainbridge Island, WA (US); William Tao Shi, Briarcliff Manor, NY (US); Francois Guy Gerald Marie Vignon, Croton on Hudson, NY (US); Emil George Radulescu, Ossining, NY (US)

(73) Assignee: KONINKLIJKE PHILIPS ELECTRONICS N.V., Eindhoven (NL)

(21) Appl. No.: 13/379,747

(22) PCT Filed: Jun. 30, 2010
(86) PCT No.: PCT/IB10/52550

§ 371 Date: Feb. 20, 2012

Related U.S. Application Data
(60) Provisional application No. 61/221,882, filed on Jun. 30, 2009.

(51) Int. Cl. A61B 8/14; A61B 8/00

(52) U.S. Cl. 600/443; 600/437

(57) ABSTRACT

Acquired echo data is corrected to reduce content from ultrasound that has undergone at least one reflection off the probe surface, for example, to reduce corresponding reverberation artefacts from imaging. In some embodiments, the propagation medium, i.e., layer or adjoining layers, through which the reverberation occurs, is, after a set of echo radiofrequency data (404, 408) has been acquired, modified in preparation for a next application of ultrasound. During that next application, it is the reverberating ultrasound signals (424), according to embodiments of the invention, that are more affected by the modification than are the non-reverberating, i.e., direct signals (420). This difference in effect is due, for example, to greater overall time of flight through the modified medium on account of reverberation in the propagation path.

The present invention relates to correcting ultrasound imaging data and, more particularly, the data arising due to reverberation of ultrasound.

Reverberation is a significant problem in many ultrasound imaging applications. This is especially the case when imaging through a layered medium in the body where strong impedance discontinuities are encountered (e.g., at bone in transcranial imaging, intercostal tissues in intercostal imaging, or at fat in abdominal imaging). It is difficult to distinguish reverberated ultrasound from signals directly backscattered from the medium of interest. The receive beamforming process does not discriminate between these two kinds of incoming signals. Reverberation artefacts manifest themselves as a high level of image clutter and as a loss of axial resolution.

The present invention is directed to overcoming or mitigating the above-described limitations of the prior art.

Aspects of what is proposed herein are based on the underlying principle that modifying the propagation medium between the probe surface and the reverberating layer affects the reverberated signals more than the direct signals, because the reverberating signals propagate between the probe and the reverberating layer more times than do the direct signals.

In an aspect of the present invention, an ultrasound apparatus is configured for, in correcting a set of echo radiofrequency data acquired from a propagation medium, taking into account at least one other set of echo radiofrequency data. These other sets are taken into account so as to reduce content from signals that reverberated through the propagation medium. The sets differ due to respective modifying of the propagation medium.

In a further aspect, the correcting includes combining, with the set to be corrected, the at least one other set.

In an aspect related to the latter, the reducing is performed by counterbalancing the content.

In one sub-aspect, the counterbalancing is against data of magnitude equal to that of the content.

In another related aspect, the combining includes time-shifting a set, from among the sets to be combined, by λ/2, λ representing wavelength.

In an alternative related aspect, the data to be combined is per-channel data or beam-summed data.

In a further related aspect, the combining includes coherent adding or coherent subtracting.

In an additional related aspect, the combining entails deriving images from the sets to be combined and averaging the derived images.

In yet another aspect, the reverberating occurs with ultrasound that reflects at least once off an outer surface of an ultrasound probe.

As one version, the apparatus comprises a wearable headpiece. The headpiece is configured for supporting an ultrasound probe and for mobility of the probe toward or away from the inside of the headpiece in modifying the propagation medium.

In a particular aspect, the modifying occurs interleavingly between the acquisitions of corresponding ones of the sets.

In a particular version, the modifying occurs during acquisition of a set.

In one aspect, the modifying causes effective propagation length to change.

In a yet different aspect, the taking into account includes making a mask based on results of comparison between a pair of the sets. The mask is for selectively excluding part of an image.

In a further aspect, the modifying includes introducing an offset to a length of a propagation path, the offset is λ/4n, λ representing wavelength, “n” being an integer that is positive or negative.

In one related sub-aspect of the above, n is equal to 1 or −1.

In one embodiment of the invention, the modifying includes inserting or removing ultrasound attenuating material.

In another embodiment, the modifying includes translating an ultrasound probe axially.

In an alternative version, the taking into account includes measuring a difference between a pair of images. Each is derived from a respective set of echo RF data.

In a yet further aspect, a data correction method includes acquiring a set of echo radiofrequency data from a propagation medium, performing one or more times in sequence the acts of modifying the medium and then acquiring a set of echo radiofrequency data, and, in correcting a set, taking into account at least one other of the acquired sets to reduce imaging artefacts arising due to reverberation that has occurred through the medium.

In a particular sub-aspect, the method includes, before the act of acquiring from the modified medium, the act of, in compensation for the modifying, offsetting a delay in the acquiring from the modified medium.

In a specific aspect, the modifying includes translating an ultrasound probe axially.

In one further aspect, ultrasound to undergo said reverberation is pulsed and bandwidth-limited. A distance between a pair of locations of an ultrasound probe axially-translated in the modifying is larger than a threshold representing half the bandwidth-limited axial resolution.

As still another aspect, an article of manufacture comprises a machine-accessible medium having instructions encoded thereon for enabling a processor to perform the above-mentioned method.

In a still further aspect, a computer software product is provided for data correction for a system having an ultrasound probe and a propagation medium. Ultrasound from the probe partially reflects to reverberate through the propagation medium. The system is used to acquire a set of ultrasound radio frequency data and to perform, one or more times in sequence, the acts of a) modifying the propagation medium; and b) using the modified system to acquire a set of ultrasound radio frequency data. The product includes a computer readable medium embodying a computer program that includes instructions executable by a processor to perform a plurality of acts. Among those acts is, in correcting a set, taking into account at least one other of the acquired sets so as to reduce content that arrived from ultrasound that reverberated through the propagation medium.

Details of the novel propagation-medium-modification-based reverberated signal elimination are set forth further below, with the aid of the following drawings.

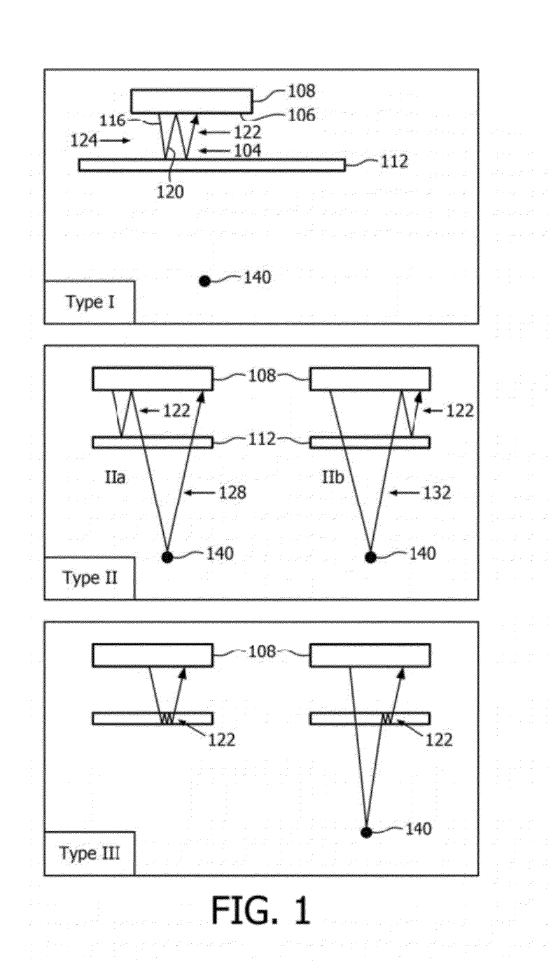

FIG. 1 is a schematic diagram illustrating some types of propagation paths of reverberating ultrasound;

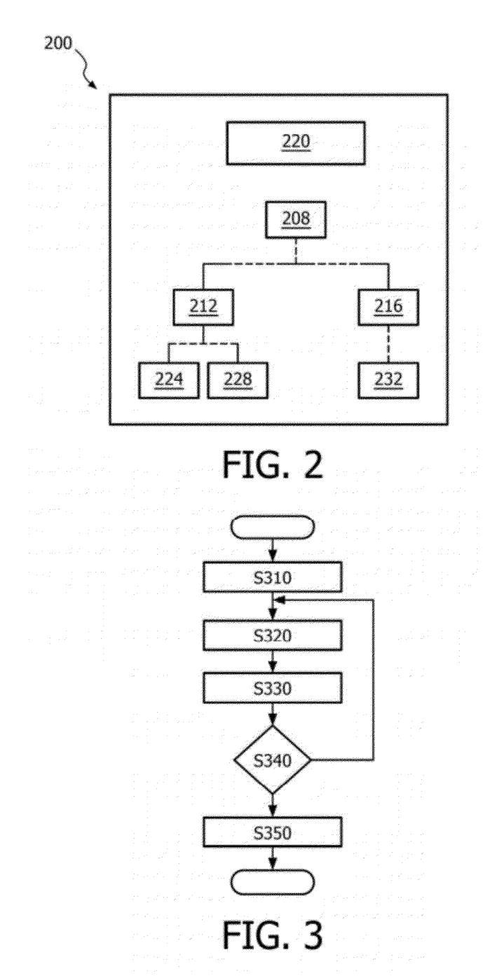

FIG. 2 is a schematic diagram depicting, as an example, an ultrasound apparatus showing mechanical connections, and information paths, between components;

FIG. 3 is a flow chart of an exemplary method of reverberation elimination;

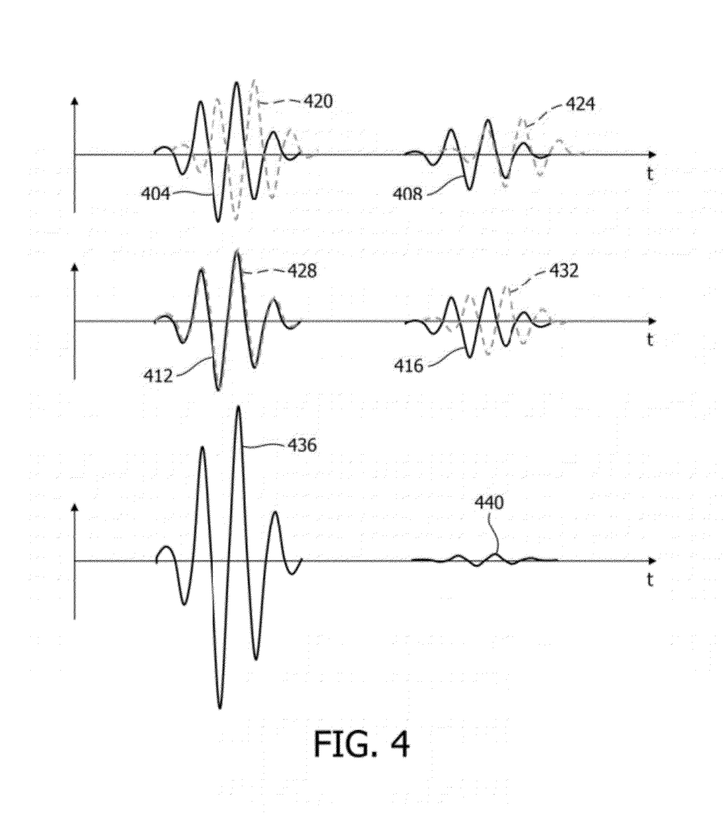

FIG. 4 is a waveform diagram indicative of a strategy for reverberated data cancellation based on a λ/4 delay in conjunction with coherent adding of acquired data sets;

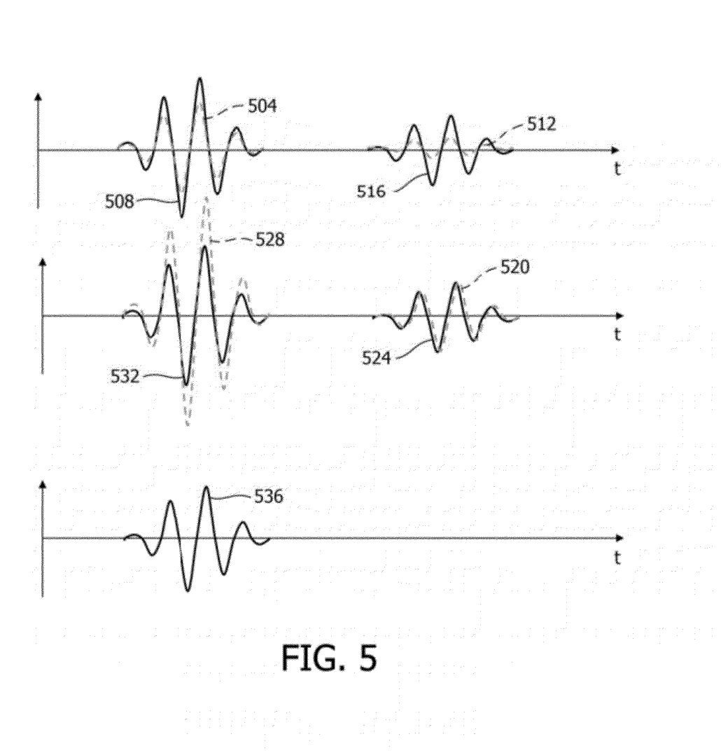

FIG. 5 is a waveform diagram indicative of an exemplary strategy for reverberation data cancellation based on inserting an ultrasound attenuating layer;

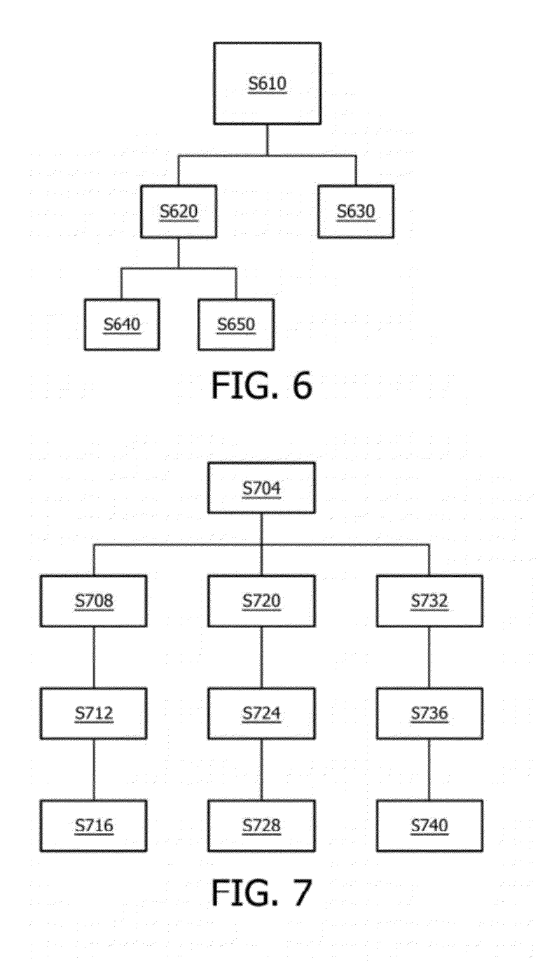

FIG. 6 is a conceptual chart showing an example of how some of the techniques and device proposed herein can be related;

FIG. 7 is a conceptual chart demonstrating, by example, different techniques used to modify the propagation medium by changing effective propagation length; and

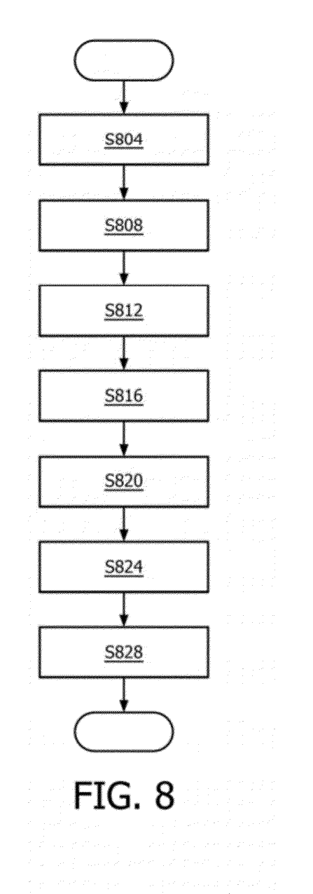

FIG. 8 is a flow chart of one example of propagation-medium-modification-based reverberated-signal cancellation through the use of a mask of cross-correlation values.

Hardware, software, and associated methods, in relation to ultrasound probe movement and its immediately adjoining propagation medium, are proposed herein for correcting acquired echo data to reduce content from ultrasound that has undergone at least one reflection off the probe surface. The reflecting ultrasound has reverberated, as between the probe surface and a reverberating layer. In some embodiments, the correcting is performed to eliminate corresponding reverberation artefacts from imaging. The “propagation medium” is defined herein as the propagation medium, or adjoining layers of propagation media, through which reverberation occurs. In some embodiments, the propagation medium is, after a set of echo radiofrequency data has been acquired, modified in preparation for a next application of ultrasound. During that next application, it is the reverberating ultrasound signals, according to embodiments of the invention, that are more affected by the modification than are the non-reverberating, i.e., direct, signals. This difference in effect is due, for example, to greater overall time of flight through the modified medium on account of reverberation in the propagation path. The difference allows reverberated signals to be distinguished, and thereby eliminated. It is noted, too, that, in some embodiments based on axial translation of an ultrasound probe to modify the propagation medium, no hardware add-ons are necessary. Instead, manual translation of the probe, complemented by signal processing, suffices.

FIG. 1 illustrates some of the propagation paths of reverberating ultrasound that occur in transcranial imaging, a type of imaging in which reverberations off a surface of a skull are unavoidable. The reverberating layer (temporal bone) is almost flat and approximately parallel to the probe surface, and most of the reverberation artefacts are confined around the normal to the probe surface.

Three types of reflections are shown: Type I, Type II and Type III. Bone, here the temporal bone, has a much greater acoustic impedance than that for the scalp soft tissue covering the bone. The bone and the soft tissue are both propagation media for the ultrasound; however, the greater the acoustic mismatch, the greater the component of the incident ultrasound that reflects at the media interface rather than refracts through the interface. Because the acoustic mismatch between bone and soft tissue is large, particularly in the case of the soft tissue covering the skull, a large component of the ultrasound reflects off the skull.

A zigzag trace 104 represents the propagation path of ultrasound that is reverberating between an outer surface 106 of the ultrasound imaging probe 108 that issued the ultrasound and a reverberating layer 112, which here is the skull. The first ray 116 of the zigzag 104 partially passes through the reverberating layer 112 as a refracted ray, and partially reflects to form the reflected ray 120. All reflecting rays of all the reverberations 122 shown in FIG. 1 have that characteristic of partial transmission and partial reflection at the point of reflection. Only the reflected components of the reflections are shown, since these are what make up the reverberating component that gives rise to reverberation artefacts to be eliminated by the devices and techniques proposed herein.

The reverberation occurs through a propagation medium 124, between the reverberation layer 112 and the probe outer surface 106. The propagation medium 124 here comprises adjoining layers. These include the scalp, and a contact medium between the probe surface 106 and the scalp, such as a gel, gel pillow or aqueous solution.

The Type I reverberations, of which trace 104 is representative, are characterized by multiple reflections between the reverberating layer 112 and the ultrasound probe 108. The reverberations result in multiple images of the reverberating layer 112 that affect the near-field of the image. The effect is adverse, because these images are not of an object of interest 140, i.e., an intended subject of ultrasound interrogation.

Type II reverberations, exemplified by traces 128, 132, involve at least one reflection off the reverberating layer and scattering by an object of interest 140 in the imaged medium. These result in multiple images of the scatterers (i.e., an image of the object 140 due to the direct signal and, according to the number of reflections, one or more other images of the scatterer 140), loss of axial resolution and clutter all across the image.

Type I and II reverberations involve at least one reflection off the imaging probe 108.

Type III reverberations, as shown by traces 136, 140, occur within the reverberating layer. Unlike type I and type II reverberations, type III reverberations do not involve reflection of sound off the imaging probe 108.

There also exist combinations of type I and III, II and III, and I and III.

A distinction can also be made among 1^{st} order, 2^{nd} order and higher order reverberations 122. 1^{st} order, type I reverberations are characterized by a single reflection off the probe surface 106. 2^{nd} order, type I reverberations correspond to two reflections off the probe surface 106, etc. Likewise, 1^{st} order, type II reverberations involve only one reflection off the probe surface 106 (whether it happens just after the transmit (type IIa) or just before the receive (type IIb). 2^{nd} order means that two reflections off the probe surface 106 are involved (i.e., twice close to the transmit, twice close to the receive, or one close to the transmit and one close to the receive), and so on. Similarly, an order can be defined for type III reverberations.

Harmonic imaging helps reduce artefacts arising from reverberations of the type IIa. Higher harmonics of the fundamental (or center) frequency of the applied ultrasound can develop due to non-linear propagation, a type of wave distortion. A harmonic double the frequency of the fundamental is received in harmonic imaging. The amplitude of the harmonic signal is, to a good approximation, proportional to the square of the amplitude of the fundamental signal, multiplied by the propagation distance. With reference to FIG. 1, the reverberated signals arising from type IIa reverberation have significantly lower amplitude that the direct signals, due to the reflections. Thus, because of the squaring effect of harmonic propagation, the harmonic component of the backscattered wave arising from the reverberated signal is even smaller compared to the harmonic component of the backscattered wave arising from the direct signal.

However, the harmonic imaging does not help to further reduce the relative magnitude of reverberations of type IIb, because the reflection occurs close to the receive after harmonic generation has occurred.

In practice, the type IIb reverberations are indistinguishable from type IIa reverberations.

Type I reverberations are also attenuated by the use of harmonic imaging, because the harmonic buildup is relatively little in the short propagation paths involved (assuming the reverberating layer 112 is close to the probe surface 106). However, harmonic imaging alone is not able to eliminate type I reverberations.

Reverberations are present in a variety of ultrasound imaging exams and most often constitute an undesirable artifact. Eliminating the impact of reverberations is of particular importance in transcranial brain imaging where the near-field of the probe 108 is cluttered by strong class I reverberations, and the rest of the image is significantly affected by class II reverberations; it is also crucial in cardiac imaging where reverberation off the ribs or on intercostals tissue contribute to overall clutter in the heart's chambers.

Preliminary in vitro observations indicate that class III reverberations (within the bone) are rather small—possibly because of the strong attenuation in the bone at diagnostic frequencies.

Reverberation orders higher than one are less problematic, because of their smaller amplitude.

The current proposal is directed to cancelling echo radiofrequency data that gives rise to class I and II, 1^{st} order reverberations, as well as higher-order reverberations.

Eliminating data tainted by reverberations is not only useful per se because it leads to better image quality. It is especially useful as an initial step in any aberration estimation strategy for aberration correction, (see U.S. Pat. No. 6,905,465 to Angelsen et al.), because it is hard to estimate an aberration-correcting delay map from per-channel signals that are significantly affected by reverberation in view of the fact that the apparent temporal waveform received on each channel is modified unpredictably. Also, removing reverberation signals is a useful step before submitting ultrasound images to automatic or semi-automatic segmentation.

FIG. 2 depicts, by way of illustrative and non-limitative example, an ultrasound apparatus showing mechanical connections, and information paths, between components.

An ultrasound apparatus 200 includes a probe 208 which, optionally, is physically connected to a propagation-medium shifter 212 or a probe axial translator 216. The broken lines denote optional inclusion, as by physical connection. The shifter 212, if connected, is further connected to ultrasound attenuating material 224 or time-delaying material 228. The translator 216 can be connected to a headpiece 232, for transcranial imaging. The probe 208, shifter 212 and translator 216 are communicatively connected to a controller 220, in a wireline or wireless connection. Any particular embodiment may vary. For example, in a freehand embodiment, in which the probe 208 is moved manually, the other hardware components 212, 216, 224, 228, 232 can be omitted.

The controller 220, which may comprise one or more integrated circuits, interacts with the receive and/or beamforming circuitry, connected to probe 208, to manipulate and to combine or otherwise take into account sets of radiofrequency (RF) data, those sets each having been acquired by the circuitry at different times. Beneficially, an effect is to reduce or cancel imaging artefacts caused by reverberation.

FIG. 3 sets forth an exemplary method 300 of ultrasound reverberation artefact elimination.

Via the receive circuitry, a set of echo RF data is acquired, incoming RF signals having been measured by the receive circuitry to create the data (step S310). This set might become a corrected set under the method 300, or may be utilized to correct one or more of the other acquired sets, as via the combining of sets, or by taking into account the set.

Next, a modify-acquire loop of the method 300 is executed one or more times.

The first step of the modify-acquire sequence is to modify the propagation medium 124 through which the reverberations 122 occur (step S320).

The second (and last) step of the modify-acquire sequence is to acquire a set of RF data from echoes that have traversed the modified propagation medium 124.

For each repeat of the modify-acquire sequence, the current step of acquisition follows the respective modifying of the propagation medium 124 in the just-previous step S320 that causes the differing among sets. In other words, the modifying occurs interleavingly between the acquisitions of corresponding ones of the sets, although, in embodiments described further below, this would not necessarily be the case (step S330).

If the modify-acquire sequence is to be repeated, processing returns to step S310. Repetition of the sequence can occur in a number of different circumstances, some examples of which are now mentioned here. For each of these circumstances, discussion in more detail appears further below. First, axial translation of the probe can be jittered, with RF acquisition at each location. The term “jittered,” in the context of axial translation, is defined herein as axially translated in an arbitrary motion. Then, a search is made for two locations with the desired axial offset. Second, the distance between any two independent probe locations can be made greater than an axial resolution threshold, in which case images derived from the sets acquired at those locations can be averaged. Third, if echo RF data representative of higher-order reverberations is to be canceled, acquisitions with corresponding modifications to the propagation medium are needed (step S340).

If the modify-acquire sequence is not to be repeated, or not to be repeated again, correction is made of a set, taking into account at least one other of the sets acquired. The taking into account may be performed by combining sets or, for example, by data set-to-data set comparison, in combination with selective spatial masking of an image, as in an embodiment described further below. The correction by combining or otherwise eliminates imaging artefacts, and the acquired data arising from ultrasound that has reverberated through the propagation medium 124 (step S350).

In a freehand embodiment to be discussed in more detail below, the modify-acquire sequence can also occur continuously (no need to physically stop the probe for frame acquisition) if, typically, the probe axial speed is much smaller than the product of the wavelength times the frame rate. Given such a speed, it is ensured that the propagation medium 124 is not modified significantly within the time needed for formation of one single image frame and corresponding RF dataset. Thus, modifying occurs during acquisition of one or more of the RF data sets, and may take the form of the inherent, unintentional motion of the user's hand on the probe 208 and/or intentional motion to modify the medium 124. In other embodiments described further below, these restrictions (interleaving motion and acquisition, or maintaining a low probe speed) can be lifted.

FIG. 4 presents waveforms indicative of a strategy for reverberation data cancellation based on a λ/4 offset in propagation length across the propagation medium 124, in conjunction with compensation for the offset, followed with coherent adding of acquired data sets, with “λ” representing wavelength. The waveforms 404, 408, 412, 416 represent echo RF signals as they are received. The horizontal axis is time, and the vertical axis is pressure amplitude. Although the waveforms 404, 408, 420, 424 share the same timeline, the paired waveforms 404, 420 relate to the timeline separately from the way in which the paired waveforms 408, 424 relate to the timeline. The undulations correspond to the generally sine-wave-like shape of an ultrasound pressure wave. The peaks correspond to compressions, and the valleys to rarefactions, in the wave. The waveforms 404, 408, 412, 416 are shown as solid to mean than they occur before modification of the propagation medium 124; whereas, the broken-line waveforms 420, 424, 428, 432 represent data acquired subsequent to that modification, i.e., from the modified medium. Moreover, the waveforms 404, 412, 420, 428, 436 on the left side represent the direct signal component of the echo RF signals being received; whereas the waveforms 408, 416, 424, 432, 440 on the right side represent the reverberated-signal component of the echo RF signals being received, for ultrasound having undergone reverberation through the propagation medium 124.

By way of example, the trace 408 on the right side of the first line represents, at any given point on its portion of the timeline, content of the set of echo RF data acquired, before modification, from signals that reverberated through the propagation medium 124.

An example of the reverberation cancellation strategy depicted in FIG. 4 involves introducing a λ/4 offset, either positive or negative, to the propagation path 116 through the propagation medium 124. One way this can be done is by axially translating the probe 208 slightly away from the skull 112. At an imaging frequency of 1.5 MHz, λ/4 is approximately equal to 0.25 millimeters (mm). Since movement is away from the skull 112, the λ/4 offset is here positive. A positive offset of sub-millimetric amplitude to the propagation path 116 can be applied by pulling the probe 208 a little away from the skull surface and using the skin resilience to maintain good contact.

In order to achieve precise 214 offsets, the ultrasound probe 208 may be mountable in a framework, e.g., plastic case, in which the transducer can be manually or automatically placed within several pre-determined positions. The framework is firmly fixed to the subject.

Alternatively, the ultrasound user can move the probe axially in a freehand, i.e., manual, fashion and a cross-correlation or similar algorithm (e.g., sum of squared differences) is applied on the RF data to identify pairs of frames that exhibit a relative offset of λ/4 in absolute value.

The subject can be a medical subject, such as a human medical patient or an animal, although the present invention is not limited to any particular living form. The subject could also be a medical sample, in vitro or ex vivo. Alternatively, the subject could be other than a life form, such as an object, the inside of which is being inspected non-invasively.

For transcranial imaging, the framework is implementable as the headpiece or helmet 232. The headpiece 232 may be adjustable to fit various head sizes, and the mounted probe 208 may be resettable for fixation in a selected one of a number of different locations, e.g., the top of the head, the right side, etc. Alternatively, or for some settings, a selection of headpieces 232 may be made available.

With respect to the effect of the translation on the set of echo RF data subsequently acquired, the structure of the set is relevant. The data accumulated in the acquisition, and held in storage, includes per-channel data and beam-summed data. Beam-summed data is data that the beamformer has summed from the various channels receiving the ultrasound beam. Per-channel data is data received on the channels but not yet summed in receive beamforming. The data start accumulating shortly after a transmit beam is emitted, and accumulate continuously during the round trip. The data set can therefore be structured, at the beam-summed level, for each A-line, as a list of sensed samples, each sample “time-stamped” in order of receipt. At the per-channel level, amplitude/phase data is time-stamped.

The axial translation away from the skull 112 has the effect of increasing, in comparison to before translation, the propagation path of a direct signal\ to any given scatterer 140.

The increase is by λ/2, due to the round trip through the propagation medium 124 modified to introduce a λ/4 offset each way.

However, reverberating signals travel more than twice through the medium 124. A 1^{st} order reverberation signal travels four times across the medium 124, increasing its round-trip propagation path by 4×λ/4=λ.

It is proposed herein to exploit the difference in offset, the offset being λ/2 for direct signals and λ for reverberated signals, to distinguish between the two types of signals.

The receive circuitry of an ultrasound device typically focuses on a particular point to be imaged by delaying the reception of echo RF data differently per channel. The delaying may be fine-tuned so that the acquired data is channel-by-channel in-phase.

To exploit the difference in offset, as between direct and reverberated signals, it is proposed herein that the entire echo RF data, directly arriving and reverberating, acquired in step 330, be time-shifted by λ/2 to compensate for the λ/2 direct-signal round-trip offset.

As focused ultrasound is emitted as an A-line and headed toward the region of interest, continuously a remaining part of it is echoed back. Upon emission, an acquisition data set accumulates the echoed data according to dynamically changing channel delays to maintain focus along the A-line. The differing channel delays change gradually in maintaining A-line focus. Accordingly, a small time-shift may be accomplished with minimal lateral error. The same holds for beam-summed data.

Operationally and by way of example, to effect the time shift, the RF content time-stamped with time t₂ assumes, by virtue of the shift, a time-stamp of t₁. The time t₁ signifies earlier acquisition than at time t₂.

The graphical manifestation of such a time shift on the set acquired after modification of the propagation medium 124 is seen in FIG. 4, where the trace 420 is rolled back in time. The extent of the time shift, i.e., the time it takes to propagate across the distance λ/2, is seen from FIG. 4 to bring the trace 420 in phase with the trace 404, as depicted by the next pair of traces 428, 412.

The physical manifestation is that coherently adding the before-modification and after-modification data sets, which adds identically-time-stamped data of the two sets, constructively adds like image content, as seen from the traces 412, 428, 436. Here, coherent adding means that each addend is in signed form. The two acquired sets will sum up constructively, as represented by the waveform 436. Recovery of essentially the pre-modification waveform is then a matter of dividing the amplitude of the waveform 436, i.e., the combined RF data, in half.

The same compensating time shift, as applied to the reverberated-signal content of the after-modification data set, does not shift far enough back to achieve constructive interference. In particular, due to the λ axial offset, the compensation brings the content of the two data sets into destructive interference, largely eliminating from the resulting combination, the reverberated-signal content, as intended. This is seen from the relevant traces 416, 432, 440. The resulting set is substantially free of the reverberated signal content.

Thus, for example, the before-modification data set is stored temporarily. The after-modification data set is also stored temporarily. In the coherent-addition embodiment, a time-shift, as described above, is applied to the temporarily-stored after-modification data set. The coherent combining is then executed.

As an alternative to time-shifting by manipulating the RF data, time-shifting can be accomplished earlier in the signal path by introducing an added common delay on the channels of the A-line. The common added delay would delay receipt of the echo on each channel by the same amount, here 212.

Specifically, in the case of pushing the probe 208 closer to the reverberating layer 112, a positive delay, i.e., postponing receipt, maintains the round trip time of flight, thereby keeping direct signals in-phase. Reverberated-signal content is destructively out-of-phase, because the time-of-flight extension falls short in compensating for the reverberating propagation.

In the other case, in which the probe 208 is pulled away from the reverberating layer 112, a negative delay is needed to shorten the round trip time of flight. Thus, the added common delay serves as a negative offset to the channel delays or to earlier activate the receive circuitry of a receive A-line.

In effect, the counterbalancing of data of equal magnitude is used as a device by which to cancel out content, of an acquired data set, from signals that reverberated through the propagation medium 124, i.e., content that otherwise would have given rise to reverberation artefacts.

A variant of this signal processing is to forego the data set time-shifting and to take the coherent difference instead of the coherent sum. The difference between the traces 404, 420 is close to the trace 436, and the difference between the traces 408, 424 to close to the trace 440. The intended scope of the invention is not limited to either coherent summing or differencing. Coherent summing could be used for some A-lines, and coherent differencing for others.

Translating the probe axially, to modify the propagation medium 124, may be achieved by motorized means or manually.

Axial translation by the probe axial translator 216 may be targeted, as by a precision motor.

Or, there is the alternative option of introducing an axial jitter to the probe 208, and identifying, by means of cross-correlation of similar technique, pairs of RF data sets that exhibit a relative 214 offset.

In particular and by way of example, the operator voluntarily, manually or automatically (by means of the probe axial translator 216) introduces an axial translation to the probe 208. This has the effect of varying the probe-to-reverberating-layer distance. Advantageously in the case of transcranial imaging, it is done without compressing the tissue of interest, i.e., the brain, because the skull is rigid. Speckle tracking, or similar phase-based techniques such as Doppler techniques, can be used on the per-channel or beam-summed RF data to estimate displacements of the probe 208 with respect to the imaged medium. The estimates of displacements are mostly based on direct echoes, because their amplitudes are the highest. Pairs of frames that exhibit the desired or target offset between the two members of the pair are automatically selected. Reverberation cancellation is then applied on each of these pairs of frames. It results in one reverberation-free image per selected pair of frames. These reverberation-free images can be averaged or displayed sequentially to achieve near-real time imaging. Alternatively, different frames with different relative displacements can be interpolated to simulate what a frame with the desired offset would look like. This embodiment relieves the need for a complicated transducer mount, without any significant adverse impact on the apparent frame rate.

In other embodiments, instead of changing the propagation length 116 between the probe 208 and the reverberating layer 112, the ultrasound attenuation can be modified. This can be done by inserting the impedance-matched attenuating material 224 over the probe surface 106. The medium shifter 212 can do this automatically, without user intervention, or the shifting can be done manually. The attenuation factor for this embodiment is, for example, a≈1/\sqrt{2}. Therefore, a wave of amplitude 1, for example, that has gone through the attenuating layer 224 has an amplitude of a≈1/\sqrt{2}. Direct echoes return with an amplitude of a²≈½ because of the round trip. 1^{st} order reverberation signals, which traverse the attenuating layer 224 four times, have an amplitude of a⁴≈¼.

To illustrate this, FIG. 5 is a waveform diagram indicative of a strategy for reverberation data cancellation based on inserting an ultrasound attenuating layer 224. The same representations of line form used in FIG. 4 apply to FIG. 5. In particular, solid lines signify before acquisition, and broken lines mean after acquisition. Likewise, the left side applies to direct signals; whereas, the right side applies to reverberated signals. Looking first at the left side, an after-modification direct-signal waveform 504 is smaller in amplitude, by a factor of a², than a before-modification waveform 508. For the reverberated-signal waveforms 512, 516, which represent 1^{st} order reverberations, the difference is more marked. They are smaller by a factor of a⁴.

Next, to take advantage of the above-described effects of the attenuating layer 224, the entire measured echo signal (direct arrivals and reverberations) is amplitude-compensated by a compensation factor of 1/a⁴.

The reverberated signal acquired after modification of the propagation medium 124 now has approximately the same amplitude as it had before modification, as evident from waveforms 520, 524.

The amplitude of the direct signal, by contrast, has been subjected to a total factor of approximately a²/a⁴=1/a²=2. Thus, the amplitude of waveform 528 is double that of waveform 532.

Next, the RF (beam-summed or per-channel) data set acquired before modification is coherently subtracted from the data set that was acquired after modification and then amplitude compensated. As a result, the 1^{st} order reverberated signals are substantially canceled; whereas, the directly arriving RF data is substantially preserved intact in the form it existed in the before-modification acquisition, as seen from trace 536.

Although the attenuation factor “a” used in the above example is ≈1/\sqrt{2}, this is not necessary. If another value is used, the direct-signal trace 536 which results from the coherent subtraction is multiplied, amplitude-wise, by a restorative factor to resemble the pre-medium-modification trace 508.

It is noted that the subtracting need not be coherent, because the counterbalancing for eliminating reverberation data does not rely on cancellation by means of adding differently signed data of comparable magnitude.

Also, as the traces 504, 508, 512, 516 demonstrate, insertion of the attenuating material 224, even without subsequent amplitude compensation and set-to-set subtraction, serves to reduce reverberation content.

In addition, the modification to the propagation medium 124 in the step S320 may entail withdrawal of the attenuating layer 224, rather an insertion of the layer. This is accompanied by swapping the amplitude compensation to the set acquired by ultrasound that has passed through the attenuating material 224.

In another embodiment, the resealing amplitude is varied until maximizing an image quality metric in a region of interest, e.g., speckle-to-cyst ratio, or until the ultrasound user is visually satisfied with the resulting ultrasound image. This is useful if the attenuation factor “a” is not known precisely. Although discussion further above of translating the probe 208 axially mentions changing round-trip propagation length as a result, a more general goal is to change effective propagation length. “Effective propagation length” is defined herein as the actual propagation length and, if time-delaying material 228 is added, an added length implied by the difference in propagation speed with and without the time-delaying material. Accordingly, instead of physically moving the transducer axially, layers of time-delaying material 228, matched in impedance with the probe surface 106 but with differing propagation velocities, can be mechanically inserted in front of the probe surface. This results in changing the effective propagation length from the probe surface 106 to the reverberating layer 112.

FIG. 6 offers an example of how some of the techniques and device proposed herein can be related. Modifying the propagation medium 124 (step S610) can be done by changing effective propagation length (step S620) or inserting/withdrawing ultrasound attenuating material 224 (step S630). Effective propagation length is changed by translating the probe 208 axially (step S640) and/or by inserting time-delaying material 228 over the probe surface 106 (step S650).

With regard to the step S640, despite the need to move the imaging probe 208 during data acquisition, pseudo real-time imaging is still achievable by triggering the axial movement of the probe with regularity, with an ECG, for instance. Several consecutive cineloops are averaged. Each corresponds to one heart beat and, at each heart beat the probe 208 is at a different position. Each cineloop includes a series of acquired frames, e.g., sets of echo RF data in the form of sample data. In the transcranial application, long averaging times can be conveniently achieved without motion artefacts, because it is possible to firmly fix the imaging probe 208 with respect to the skull 112. The averaging is not necessarily between the cineloops at one position and the cineloop at the different position. It may be, for instance, that all images of the current cineloop are combined with the most recent images at the other position.

In the embodiment where the probe is translated manually and a cross-correlation (or similar) routine automatically identifies pairs of images acquired with the two probe locations being offset by λ/4, the current frame is associated with the most recent frame affording the λ/4 offset, for correction of the current frame.

FIG. 7 demonstrates, by example, different techniques used to modify the propagation medium by changing effective propagation length.

As mentioned in connection with the step 620 in the previous chart, changing the effective propagation length (step S704) may entail translating the probe 208 axially (step S630) and/or inserting/withdrawing ultrasound material 228 of differing velocity (step S650).

The technique of introducing a λ/4n offset to the effective propagation length will first be discussed below in the context of n=1, which is the case of 1^{st} order reverberation through the propagation medium 124.

As discussed above in connection with FIG. 4, a λ/4 offset may be introduced (step S708), compensated by a λ/2 delay (step S712). Combination is by coherent adding or, with the compensation foregone, by coherent subtraction. Odd-order reverberated signals are thus largely eliminated (depending on the pulse length and shape and on the order of the reverberation) but even order reverberations are not. Importantly, 1^{st} order reverberations, which are the most prominent, are eliminated (step S716).

A general method, to go beyond eliminating 1^{st} order reverberated signals, is to selectively eliminate n^{th} (even or odd) order reverberated content. This is done by introducing a λ/4n offset and compensating by λ/2, followed by coherently summing the data. In other embodiments, cancelling reverberation artefacts is achievable, without the need for precise counterbalancing, by averaging images derived from respective ones of the sets. A plurality of images of the imaged medium are acquired corresponding to N probe-tissue distances. The spatial distance between two independent probe locations is set to be larger than a threshold Δr/2. The parameter Δr/2 represents half the pulse length that determines the axial resolution of the image, i.e., the bandwidth-limited axial resolution of the transducer. The parameter Δr=2c/B, where c is the speed of light, and B is the bandwidth, a frequency range centered about the center frequency of the imaging pulse. In the case of type I or II reverberations, the difference in round-trip time of flight between direct signals and signals that have reverberated through the propagation medium 124 varies with the distance between the ultrasound probe 208 and the reverberating layer 112 (off of which the reverberations occur with the probe surface 106). Averaging the N different resulting images, preferably but not necessarily after time-compensation so that the direct echoes arrive at the same time in all considered frames, thus increases the direct signal/reverberations ratio. This is accomplished by spreading the reverberations over an extended axial range, and keeping the direct signals' arrival time constant. The averaging of images can be performed coherently or incoherently. If the spatial distance between the N axial positions of the transducer is greater than the threshold Δr, then the signal-to-reverberation clutter ratio is theoretically increased by a factor of N. In this embodiment, axial translations of the probe on the order of several millimeters may be needed. If the skin resilience is insufficient to maintain good contact all along the transducer's axial trip, the coupling medium between the probe and the skull surface can be made of a water or gel pillow. Alternatively, layers of time-delaying material 228 can be mechanically inserted in front of the probe surface 106. Advantageously, no precise control of the probe location is required. The offset, for this embodiment, between independent probe locations the corresponding data for which is to be averaged exceeds Δr (step S720). Images are derived from the acquisition sets to be combined by virtue of the averaging of images. Before the averaging, the RF data sets acquired with different probe locations are temporarily stored in memory. They are then respectively time shifted to bring the sets mutually into registration, with respect to direct echo data (step S724). The averaging of the images derived from time-shifted data sets can be performed coherently or incoherently (step S728).

A variant to controlled axial translation of the probe 208 is the introduction of an axial jitter and the subsequent use of speckle tracking or Doppler-like techniques to estimate the relative axial displacement of the probe between two frames. Then, frames that are further apart than one axial resolution length are combined.

As a further alternative, a difference between a pair of sets of RF data acquired in the acquisition steps S310, S330 is measured. This alternative method is based on the assumption that direct-signal content is more energetic and thus predominates over reverberated-signal content, an assumption that usually holds.

In particular, propagation-medium-modification-based reverberated-signal cancellation can also be effected through the use of a mask of correlation values.

In this embodiment, the probe is, likewise, translated axially (step S732). A mask is made based on the results of cross-correlation of two of the echo RF data sets (step S736). Then, the mask is applied to a data set to mask out areas of low correlation, indicative or reverberation/multipath (step S740).

In this technique, the probe 208 is translated axially, manually or with a motorized device, but not necessarily in a very controlled way, and by amounts that can exceed the wavelength order of magnitude.

Details of one example of propagation-medium-modification-based reverberated-signal cancellation through the use of cross-correlation are set forth below in correspondence with FIG. 8.

An echo RF data set is acquired (step S804), and the probe 208 is translated axially by a distance x (step S808), which can be either known precisely as with the use of a spatial translator device, or estimated through speckle tracking or Doppler techniques, or other cross-correlation or similar based-techniques. Another data set is then acquired (step S812). The latter set is then time-shifted by 2x to put direct-signal content into approximate registration with that of the previously-acquired set, as described above in connection with other embodiments. The reverberated-signal contents of the respective sets are now misaligned (step S816).

Then, instead of coherently adding the two sets, the same image regions derived respectively from the two sets are cross-correlated (or compared using a similar technique such as sum of squared differences, etc.) (step S820). The end product is a matrix of coefficients each of which corresponds to a respective pixel representing an associated depth and A-line. Each pixel is obtained by cross-correlating (or otherwise comparing) RF data from a region of interest of limited extent around the depth and direction/azimuth of the corresponding spatial pixel location, e.g., two wavelengths prior and posterior in the axial dimensions and one A-line laterally in the transverse direction(s). The direct-signal-based pixels will have correlation coefficients significantly higher than the reverberated-signal-based pixels, because the direct signals are realigned whereas the reverberated signals are not. Areas of the image that exhibit low correlation coefficients will be areas dominated by reverberation or multipath. Image areas associated with high echo intensity values and low correlation coefficients are most likely to come from reverberations and thus need to be tagged or eliminated.

A mask is created, using the results of the cross-correlation. The mask has high values where the cross-correlation coefficients are high (indicating dominant presence of direct signals) and low values where the cross-correlation coefficients are low and especially if associated with high echo intensity values (indicating dominant presence of reverberated signals). In making the mask, any negative coefficients are set to zero (step S824). In effect, correction of an acquired one of the RF sets is performed by taking into account one or more of the other RF sets, which, in the instant embodiment, entails making, based on results of comparison between a pair of the sets, a mask for selectively excluding part of an image. The mask is applied to the finally displayed image, and can be spatially smoothed to optimize display quality (step S828).

In effect, in correcting a set of echo radiofrequency data, taking into account at least one other set of echo radiofrequency data so as to reduce content from signals that reverberated through the propagation medium 124 includes making a mask based on results of comparison between a pair of sets from among the set and the at least one other set. The mask is for selectively excluding part of an image.

With reference back to the time-shifting step S8165, if the distance x is not precise or known or is estimated through cross-correlations, the time-shifting step S816 is not executed. Instead of executing a post-time-shifting, in-place cross-correlation, a cross-correlation search is performed. It is noted that motion of the probe 208 is not confined to periods between data set acquisition. Motion may be performed during the acquisition.

The cross-correlation kernel size can be, for example, two wavelengths before and two wavelengths after. If searching, the searching window size should encompass the amount of axial translation performed. The kernel size and window searching size may be dynamically set, so that they may increase based on the cross-correlation results.

Finally, the mask is used as a multiplying mask to the original brightness images, thus multiplying direct signals with high values and reverberated signals with low values. Alternatively, it may be chosen to tag the pixels of the brightness maps which have corresponding values on the correlation mask that are lower than a certain threshold, by displaying them with a different color scale. (Thus, no information is hidden to the user, but it helps the user to identify which parts of the image come from real physical structures and which are reverberation artefacts).

The cross-correlation embodiment advantageously is easy to implement and robust. There is no need for precise axial translations, and the algorithm is wavelength-independent.

In all embodiments that involve time-shifting of received data, a first refinement, beyond mere time-shifting, entails modifying the scan's apex position for receive beamforming. This is done so as to keep the image apex at the same physical location when the probe 208 is translated. Another refinement would be to also adjust the transmit beam shape so that the direct ultrasound field sensed at any point within the imaged medium would be the same, whatever the probe-to-tissue distance.

The inherent nature of the innovative proposed methods and devices herein make them suitable not only for transcranial ultrasound imaging where strong reverberations are unavoidable, but also for all applications suffering from unwanted reverberation or multipath effects.

It should be noted that the above-mentioned embodiments illustrate rather than limit the invention, and that those skilled in the art will be able to design many alternative embodiments without departing from the scope of the appended claims. For example, higher-order reverberated content can be canceled if modification of the propagation medium 124 is by ultrasound attenuation. In particular, amplitude compensation for ultrasound attenuating layers may be tailored to selectively eliminate n^{th} order reverberated signals by resealing the signal by 1/a²ⁿ. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer having a computer readable medium. The mere fact that certain measures are recited in mutually different dependent claims does not indicate that a combination of these measures cannot be used to advantage.

1. An ultrasound apparatus (200) configured for, in correcting a set of echo radiofrequency data acquired from a propagation path through a propagation medium, taking into account at least one other set of echo radiofrequency data received from the propagation medium so as to reduce content from signals that reverberated through said medium, said set and said at least one other set all differing due to respective axially modifying the propagation path of said medium.

2. The apparatus of claim 1, wherein said correcting comprises combining, with said set to be corrected, said at least one other set.

3. The apparatus of claim 2, the reducing being performed by counterbalancing said content.

4. The apparatus of claim 3, said counterbalancing being against data of magnitude equal to that of said content.

5. The apparatus of claim 2, said combining comprising time-shifting a set, from among the sets to be combined, by λ/2, λ representing wavelength.

6. The apparatus of claim 2, the data to be combined, in said combining, being at least one of per-channel data and beam-summed data.

7. (canceled)

8. The apparatus of claim 2, said combining comprising deriving images from the sets to be combined and averaging the derived images.

9. (canceled)

10. The apparatus of claim 1, comprising a wearable headpiece, said headpiece having an inside and being configured for supporting an ultrasound probe and for mobility of said probe at least one of toward and away from said inside in performance of said modifying.

11. (canceled)

12. (canceled)

13. The apparatus of claim 1, said modifying causing effective propagation length to change.

14. The apparatus of claim 1, said taking into account comprising making, based on results of comparison between a pair of sets from among said set and said at least one other set, a mask for selectively excluding part of an image.

15. The apparatus of claim 1, said modifying comprising introducing an offset to a length of a propagation path, said offset being λ/4n, λ representing wavelength, “n” being an integer that is positive or negative.

16. The apparatus of claim 15, n being equal to 1 or −1.

17. The apparatus of claim 1, said modifying comprising at least one of inserting and removing ultrasound attenuating material.

18. The apparatus of claim 1, said modifying comprising translating an ultrasound probe axially.

19. (canceled)

20. A data correction method for correcting for reverberation artifacts in echo signals received by an ultrasound probe comprising:
acquiring a set of echo radiofrequency data from a propagation medium;
performing one or more times in sequence the acts of modifying said medium by axial change of a propagation path through the propagation medium and then acquiring a set of echo radiofrequency data; and,
in correcting a set from among the acquired sets, taking into account at least one other of the acquired sets to reduce imaging artifacts arising due to reverberation that has occurred through said medium.

21. The method of claim 20, further comprising, before the act of acquiring from the modified medium, the act of, in compensation for said modifying, offsetting a delay in said acquiring from the modified medium.

22. The method of claim 20, said modifying comprising translating an ultrasound probe axially, said translating comprising imparting a jitter to said probe.

23. The method of claim 20, ultrasound to undergo said reverberation being pulsed and bandwidth-limited, a distance between a pair of locations of an ultrasound probe axially-translated in said modifying being larger than a threshold representing half the bandwidth-limited axial resolution.

24.-25. (canceled)
